(19) United States
(12) Patent Application Publication
(10) Pub. No.: US 2026/0248755 A1
(43) Pub. Date: Aug. 27, 2026

(54) EFFICACIOUS INCREASE OF KETONE LEVELS IN A HUMAN BODY AND SYNTHESIS OF NOVEL COMPOSITIONS OF KETONE BODY ANALOG ESTERS FOR USE AS A DIETARY SUPPLEMENT

(71) Applicant: Ketone Catalyst Innovations, LLC, Ponte Vedra Beach, FL (US)

(72) Inventors: Daryl Richard Sauer, Trevor, WI (US); Anthony Capasso, Ponte Vedra Beach, FL (US)

(73) Assignee: Ketone Catalyst Innovations, LLC, Ponte Vedra Beach, FL (US)

(21) Appl. No.: 19/546,853

(22) Filed: Feb. 23, 2026

Related U.S. Application Data
(60) Provisional application No. 63/762,207, filed on Feb. 24, 2025.

(51) Int. Cl. A61K 31/22

(52) U.S. Cl. CPC A61K 31/22

(57) ABSTRACT

Methods of efficaciously increasing ketone body levels in a human bloodstream, thereby supporting various health benefits associated with ketosis, including improved metabolic health and enhanced endurance performance. Compositions including mixtures of esters that are used by a user's body to create ketosis related molecules in the blood stream are disclosed. Methods of forming the mixtures and exemplary starting materials are disclosed. Compositions may comprise mixtures of ketone body analog esters designed for nutritional supplementation. Compositions include esters such as 3-Hydroxy-3-methylbutyl 3-oxobutanoate and(S)-3-Hydroxybutyl 3-oxobutanoate, which mimic the physiological effects of natural ketosis. The present invention includes methods for synthesizing compositions through reactions involving 1,3-Butanediol, 3-Hydroxy-3-methylbutanol, and various acids in the presence of enzyme systems. Resulting esters enhance energy metabolism, support muscle recovery, promote cognitive function, and aid in weight management. Compositions may be tailored by adjusting reactant ratios and reaction conditions, offering flexibility in achieving desired ester profiles.

### CROSS REFERENCE TO RELATED APPLICATIONS

This application claims priority to U.S. Provisional Application No. 63/762,207, filed Feb. 24, 2025, entitled EFFICACIOUS INCREASE OF KETONE LEVELS IN A HUMAN BODY AND SYNTHESIS OF NOVEL COMPOSITIONS OF KETONE BODY ANALOG ESTERS FOR USE AS A DIETARY SUPPLEMENT, the entire disclosures of which are incorporated herein by reference.

### FIELD OF THE INVENTION

The present invention relates to methods of efficaciously increasing ketone body levels in a human the synthesis of novel compositions, more specifically compositions comprising mixtures of ketone body analogs for use as nutritional supplements.

### BACKGROUND OF THE INVENTION

Ketone bodies are metabolites that the human body forms through the breakdown of fats during times of low food intake or other health related issues. Three major ketone bodies are acetoacetate, β-hydroxybutyrate, and acetone. Acetoacetate and β-hydroxybutyrate will be further metabolized to generate energy for the body, with acetone being a by-product. Ketone bodies can be externally supplemented to achieve benefits without the breakdown of fats. Besides the use of ketone bodies as an energy source there are more health benefits, such as helping with weight loss, aging, diabetes, Alzheimer's, Parkinson's, seizures, cancer, psychiatric disorders, and issues dealing with irregular glucose metabolism. Ketone body esters, which are metabolically bioavailable versions of ketone bodies, have been shown in supplement form to encompass the beneficial aspects of ketone bodies.

In natural Ketosis three primary ketone bodies may be produced and released into the bloodstream: acetoacetate (AcAc), beta-hydroxybutyrate (BHB), and acetone and exist in the bloodstream at the same time. Acetoacetate is the first ketone body synthesized in the liver. It is either converted to beta-hydroxybutyrate, the most abundant and stable ketone body in the blood, or it spontaneously breaks down into acetone. Acetone, being volatile, is exhaled through the breath, giving rise to the characteristic “fruity” odor associated with ketosis.

Beta-hydroxybutyrate and acetoacetate circulate in the bloodstream and are readily taken up by tissues, including the brain, muscles, and heart, where they are converted back into acetyl-CoA for use in the energy-producing citric acid cycle. The presence of these ketone bodies in the blood is a hallmark of ketosis and provides a reliable indicator of the body's metabolic state.

Supplementary use of molecules that are related to the molecules produced during Ketosis can be made with ketone body related molecules and esters can have beneficial effects. For example, small quantities of Hydroxy β-methylbutyrate molecules are available from natural sources such as catfish, aged meats, grapefruit and corn; however, the small quantities make it difficult to have a substantial effect. The use of β-hydroxy β-methylbutyrate as a supplement has been shown to have health benefits including activation of the mTOR kinase, which initiates protein synthesis to help build and repair muscles. The supplementation of β-hydroxy β-methylbutyrate has also been shown to prevent protein degradation. Preventing muscle loss and promoting the repair and development of muscles is important, especially in a variety of the population including athletes and the aging.

A supplement regime that provides ketosis related molecules or molecules that are used by the body to produce ketosis related molecules in quantities similar to those produced during natural ketosis would be desirable.

### BRIEF DESCRIPTION OF THE DRAWINGS

The accompanying drawings, which are incorporated in and constitute a part of this specification, illustrate several examples of the invention and, together with the description, serve to explain the principles of the invention.

FIGS. 1A-1C illustrate exemplary chemical reactions between 1,3-Butanediol and various acids according to the present disclosure.

FIGS. 1D-1F illustrate exemplary chemical reactions between 1,3-Butanediol and various acids according to the present disclosure.

FIG. 2A illustrates an exemplary mixture of reactants according to the present disclosure.

FIG. 2B illustrates another exemplary mixture of reactants according to the present disclosure.

FIG. 3A illustrates a product mixture composition related to the reactants of FIG. 2A.

FIG. 3B illustrates a product mixture composition related to the reactants of FIG. 2B.

FIG. 4A illustrates an exemplary mixture of compounds resulting from metabolism of the composition of FIG. 3A.

FIG. 4B illustrates an exemplary mixture of compounds resulting from metabolism of the composition of FIG. 3B.

### SUMMARY OF THE INVENTION

Accordingly, the present invention provides a description of methods to synthesize compositions that simultaneously provide multiple chemical compounds related to natural ketosis.

The present invention provides a method of synthesis combining a number of starting materials that together generate controllable compositions of these product molecules.

In some embodiments, a combination including two or more of the following ketone esters may be formed for nutritional ingestion by a patient. The combination may include two or more of the following: 3-Hydroxy-3-methylbutyl 3-oxobutanoate, (S)-3-Hydroxybutyl 3-oxobutanoate, (R)-3-Hydroxybutyl (S)-2-hydroxypropanoate, 3-Hydroxy-3-methylbutyl (S)-2-hydroxypropanoate, (R)-3-Hydroxybutyl (S)-3-hydroxybutanoate, and 3-Hydroxy-3-methylbutyl (S)-3-hydroxybutanoate. In some embodiments, the combination may also include secondary esters that may form in lower concentrations due to steric interactions at the secondary hydroxide locations.

In some embodiments, a combination of the esters may be formulated by reacting a combination of reactant molecules in a single reaction where the various esters may result from the random interactions between two different reactants. The relative amount of reactant products may be controlled by adjustments in the relative amounts of starting materials and by controlling the reaction conditions, such as temperature, enzyme choice, and the like.

Asymmetric centers exist in the compounds of the present invention and are designated by the letters “R” or “S,” depending on the configuration of substituents around the chiral carbon atom. It should be understood that the invention encompasses all stereochemical isomeric forms, both enantiomerically pure and any mixture thereof and also the designation of a particular stereoisomer, except as specifically noted in the claims, is intended to cover a similar discussion for other stereoisomers.

### DETAILED DESCRIPTION OF THE INVENTION

The present invention provides compositions and methods for synthesizing and providing supplementation with ketone body analog esters. The compositions may include mixtures of esters such as 3-Hydroxy-3-methylbutyl 3-oxobutanoate, (S)-3-Hydroxybutyl 3-oxobutanoate, and (R)-3-Hydroxybutyl (S)-2-hydroxypropanoate, among others. These esters mimic the effects of natural ketosis, providing benefits such as enhanced energy metabolism, muscle recovery, cognitive function, and weight management.

The methods of synthesis involve reacting compounds such as 1,3-Butanediol and 3-Hydroxy-3-methylbutanol with various acids in the presence of enzyme systems, allowing for the creation of specific ester profiles by adjusting reactant ratios and reaction conditions. Additionally, the invention includes methods of providing supplementation by administering these compositions to users, thereby increasing ketone body levels in the bloodstream, and supporting overall health and wellness. The invention offers a versatile approach to achieving the physiological benefits of ketosis through external supplementation.

Elements of the present invention may be better understood from the following description of the preferred embodiments. The preferred embodiments are intended to provide a description of the present invention and are not intended to limit the invention in any manner.

The present disclosure relates to a class of reactions and their resulting products that result in a combination of ester products in a product stream. In some embodiments, the resulting mixture may include a predominance of desired esters intended to create physiological effects, such as a mimicking of the resultant blood concentration of various ketone bodies after ingestion that also result in a natural ketosis state.

### Definitions

The following explanations of terms and methods are provided to better describe the present disclosure and to guide those of ordinary skill in the art in the practice of the present disclosure.

“1,3-butanediol” refers to 1,3-butanediol in any stereochemical form, including (R)-1,3-butanediol, (S)-1,3-butanediol, racemic mixtures, or enantiomerically enriched compositions thereof.

“3-hydroxy-3-methylbutanol” refers to 3-hydroxy-3-methyl-1-butanol in any stereochemical form, including racemic mixtures and enantiomerically enriched forms.

“about” or “approximately,” when referring to a numerical value, mean±10% of the stated value unless otherwise specified.

“Acetoacetyl” refers to the functionality with the formula CH₃COCH₂CO—.

“acetoacetic acid” refers to 3-oxobutanoic acid and includes salts, solvates, and stabilized forms thereof unless otherwise specified.

“administering” refers to delivering a composition to a user by any suitable route, including oral, sublingual, buccal, enteral, parenteral, topical, transdermal, or inhalational routes. In certain embodiments, administering comprises oral ingestion of a liquid, capsule, powder, suspension, or softgel formulation.

“aid in weight management” refers to supporting metabolic pathways associated with energy utilization, fat oxidation, or appetite regulation.

“beta-hydroxybutyric acid” or “β-hydroxybutyric acid” refers to 3-hydroxybutanoic acid and includes (R)-beta-hydroxybutyric acid, (S)-beta-hydroxybutyric acid, racemic mixtures, salts, and solvates.

“β-Hydroxybutyryl” refers to the functionality with the formula CH₃CH(OH)CH₂CO—.

“combining” refers to bringing two or more components into contact under conditions sufficient to permit reaction, including simultaneous addition, sequential addition, staged addition, continuous feeding, or in situ generation of one or more components.

“comprising” is intended to be open-ended and means including but not limited to. “Consisting of” and “consisting essentially of” have their customary patent law meanings.

“composition comprising esters” refers to a composition containing one or more ester compounds, optionally in combination with unreacted starting materials, byproducts, excipients, carriers, solvents, stabilizers, or additional active ingredients. The composition may be a reaction mixture, purified product, partially purified product, or formulated end-use product.

“Derivative” refers to a compound or portion of a compound that is derived from or is theoretically derivable from a parent compound.

“enhance muscle recovery” refers to supporting physiological processes associated with post-exertion repair, metabolic replenishment, or reduction of fatigue markers.

“enzyme system” refers to one or more enzymes capable of catalyzing esterification, transesterification, or acyl transfer reactions between alcohol and acid substrates. The enzyme system may include lipases, esterases, cutinases, acyltransferases, or functional variants thereof.

“Ester” is represented by the formula —OC(O)R, where R can be any organic group, typically an alkyl group. In some embodiments, “ester” refers to a compound formed by reaction of an alcohol functional group and a carboxylic acid functional group, including monoesters, diesters, mixed esters, and partially esterified species. The term encompasses compounds formed by enzymatic esterification, transesterification, or acyl transfer reactions.

“Esterification” refers to the reaction of an alcohol with a carboxylic acid or a carboxylic acid derivative to give an ester.

“hydroxy” is represented by the formula —OH.

“immobilized enzyme” refers to an enzyme attached to, entrapped within, adsorbed onto, or otherwise associated with a solid support, including polymeric resins, silica, magnetic particles, membranes, or other matrices, such that the enzyme may be retained and optionally reused following reaction.

“increase ketone body levels” refers to elevating circulating concentrations of ketone bodies in the bloodstream, including beta-hydroxybutyrate and/or acetoacetate, relative to baseline levels.

“lactic acid” refers to 2-hydroxypropanoic acid and includes (S)-lactic acid, (R)-lactic acid, racemic mixtures, salts, and solvates.

“mixture” refers to a composition containing two or more distinct chemical species, including mixtures of different ester compounds, mixtures of stereoisomers, or mixtures of reaction products and starting materials.

“oxo,” as used herein, represents ═O.

“Polymer” refers to a molecule that has repeating units called monomers and, as used herein, represents repeating units of acetoacetyl or β-Hydroxybutyryl functionalities.

“promote cognitive function” refers to supporting normal neurological processes including mental clarity, focus, or cognitive performance.

“promote overall wellness” refers to supporting one or more physiological systems contributing to general health.

“reacting” refers to subjecting two or more chemical species to conditions under which a chemical transformation occurs, including enzymatic catalysis, resulting in formation of one or more ester products.

“stereochemically enriched” refers to a composition having an enantiomeric excess (ee) greater than 50%, greater than 75%, greater than 90%, or greater than 95% of one enantiomer relative to another.

“supplementation” refers to administration of a composition intended to support normal physiological function, metabolic pathways, energy production, or overall wellness.

“support cardiovascular health” refers to supporting normal cardiac and vascular physiological function.

“support energy metabolism” refers to providing substrates or intermediates capable of participating in metabolic pathways associated with ATP production, mitochondrial oxidation, or related bioenergetic processes.

“support immune function” refers to supporting normal immune system processes.

“Therapeutically acceptable” refers to those compounds (or salts, zwitterionic forms, esters, polymers, etc.) that are suitable for the treatment of diseases without undue toxicity, irritation, or allergic response, are commensurate with a reasonable benefit/risk ratio, and are effective for their intended use.

“Transesterification” refers to the chemical exchange of functionalities between an ester and an alcohol to form new esters and alcohols.

“user” refers to a human subject and may include individuals seeking nutritional supplementation, metabolic support, athletic performance enhancement, recovery support, cognitive support, cardiovascular support, immune support, or weight management. In some embodiments, the user may be a mammal.

While the above definitions are used in this application to describe how one of ordinary skill in the art may generally understand the terms used to describe the invention, the definitions are not intended to limit the definition of the above terms in any manner. It is understood that the terms may have a broader meaning than set forth above to those of skill in the art. The terms as used to describe the present invention are meant to provide a general understanding of when the terms may be used and enablement of the present invention.

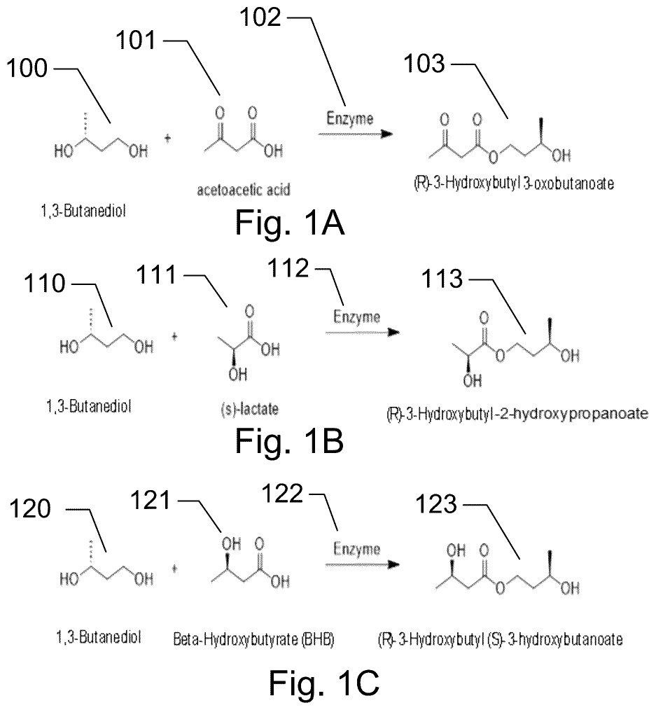

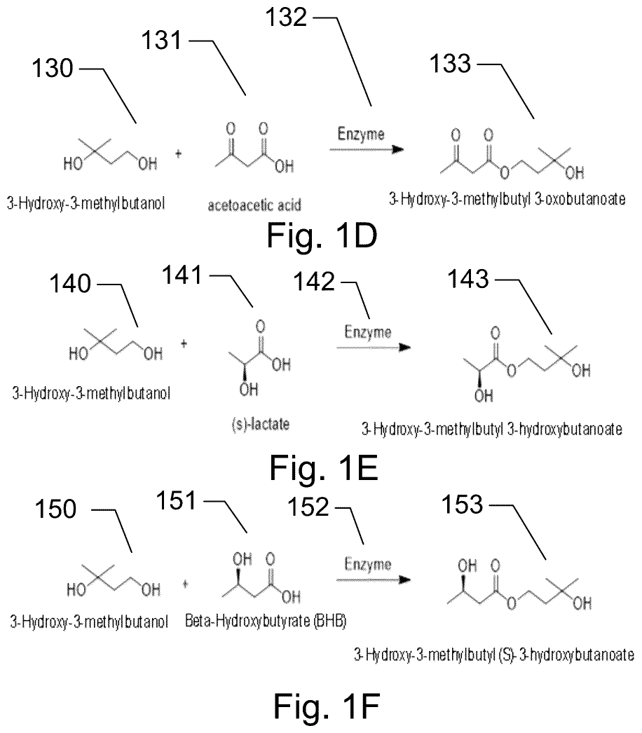

Referring now to FIG. 1A, an exemplary reaction of 1,3 Butanediol 100 with acetoacetic acid 101 and an enzyme system 102 is illustrated, resulting in a product of (R)-3-hydroxybutyl-3-oxobutanoate 103.

Referring now to FIG. 1B, an exemplary reaction of 1,3 Butanediol 110 with lactic acid 111 and an enzyme system 112 is illustrated, resulting in a product of (R)-3-hydroxybutyl-2-hydroxypropanoate 113.

Referring now to FIG. 1C, an exemplary reaction of 1,3 Butanediol 120 with Beta-Hydroxybutyric acid 121 and an enzyme system 122 is illustrated, resulting in a product of (R)-3-hydroxybutyl (S)-3-hydroxybutanoate 113.

Referring now to FIG. 1D, an exemplary reaction of 3-Hydroxy-3-methylbutanol 130 with acetoacetic acid 131 and an enzyme system 132 is illustrated, resulting in a product of 3-hydroxy-3-methylbutyl-3-oxobutanoate 133.

Referring now to FIG. 1E, an exemplary reaction of 3-Hydroxy-3-methylbutanol 140 with lactic acid 141 and an enzyme system 142 is illustrated, resulting in a product of (R)-3-hydroxybutyl-2-hydroxypropanoate 143.

Referring now to FIG. 1F, an exemplary reaction of 3-Hydroxy-3-methylbutanol 150 with Beta-Hydroxybutyric acid 151 and an enzyme system 152 is illustrated, resulting in a product of 3-hydroxy-3-methylbutyl (S)-3-hydroxybutanoate 113.

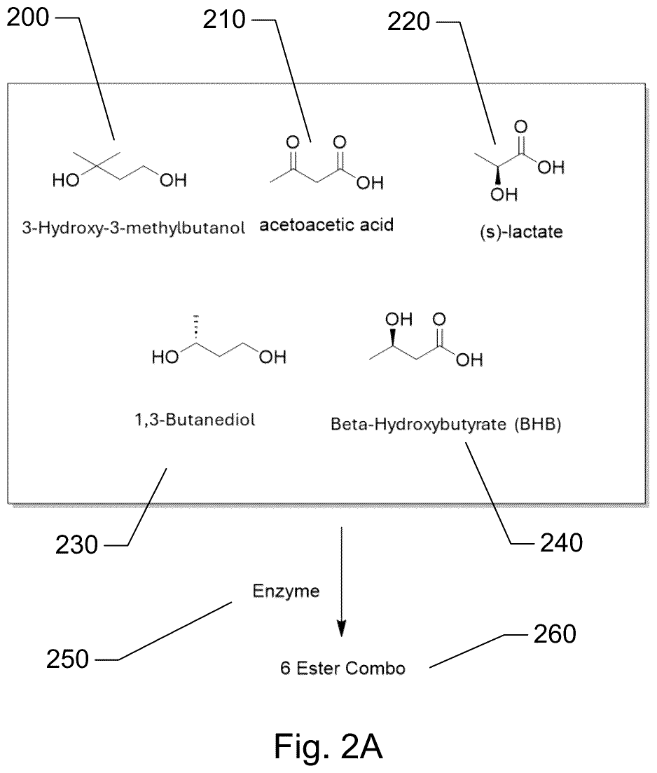

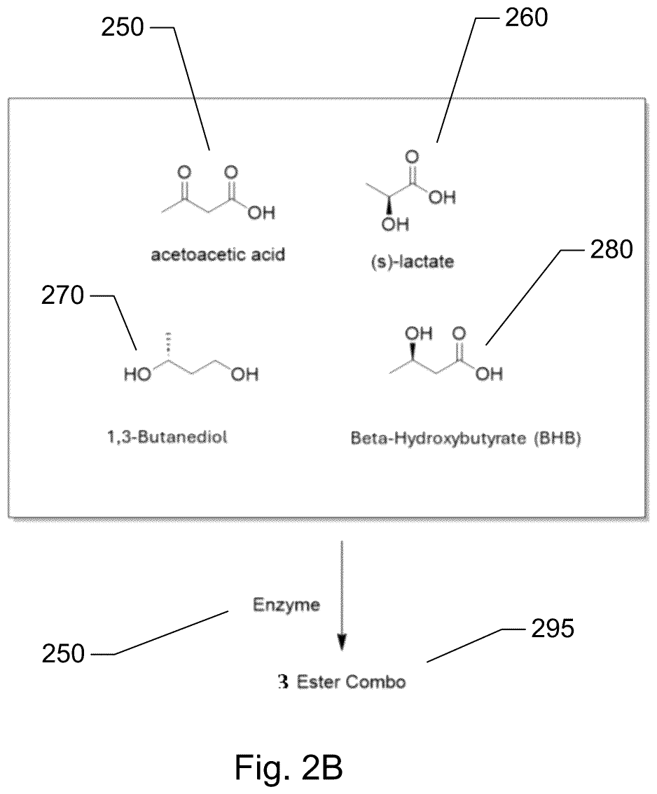

In some embodiments, combinations of the reactants of these six exemplary reactions may efficiently be used to form mixtures of target ester molecules for various compositions. For example, in reference to FIG. 2A, a production process may involve processing a mixture of starting chemicals as described below where the molecules may be dissolved in solvent systems, which may include, but is not limited to, one or more of: water, ethyl acetate, toluene, and xylene as examples.

The solution may be heated. The reaction may typically be performed above room temperature and up to 150° C. While the preferred temperature for the reaction may be in the range of 20° C. to 150° C., it should be understood that reaction may take place above 150° C. and would likely provide for a faster reaction and reduce the byproducts of the reaction. The reactions may also be catalyzed with acids or bases such as sulfuric acid or triethylamine but are not limited to these compounds. The reaction may also be conducted with an enzyme, more specifically with a lipase in either the free form or immobilized on a solid support. The purification of these reactions to isolate the desired products may include column chromatography, distillation, extraction, and other more complex purification methods.

Referring again to FIG. 2A, the mixture may include 3-Hydroxy-3-methylbutanol 200, acetoacetic acid 210, lactic acid 220, 1,3-Butanediol 230, and Beta-Hydroxybutyrate 240. Together with an enzyme system 250. The resulting product may include 6 targeted primary esters 260 in some embodiments.

Referring to FIG. 2B, an exemplary mixture for reaction with fewer reactants is illustrated. In some embodiments, more or less reactants may be utilized. The mixture may include acetoacetic acid 250, lactic acid 260, 1,3-Butanediol 270, and Beta-Hydroxybutyric acid (or its butyrate salt) 280 together with an enzyme system 290 to result in a product that may include 3 targeted primary esters 295.

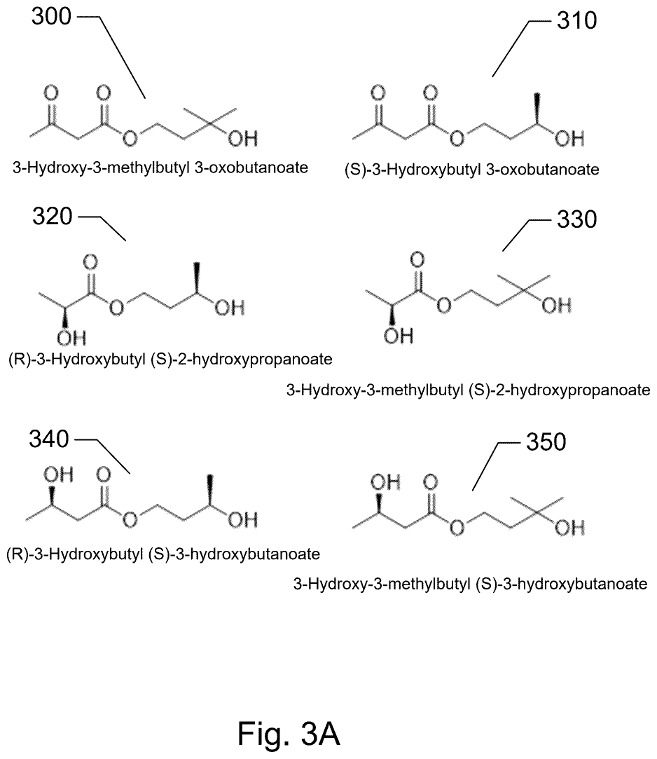

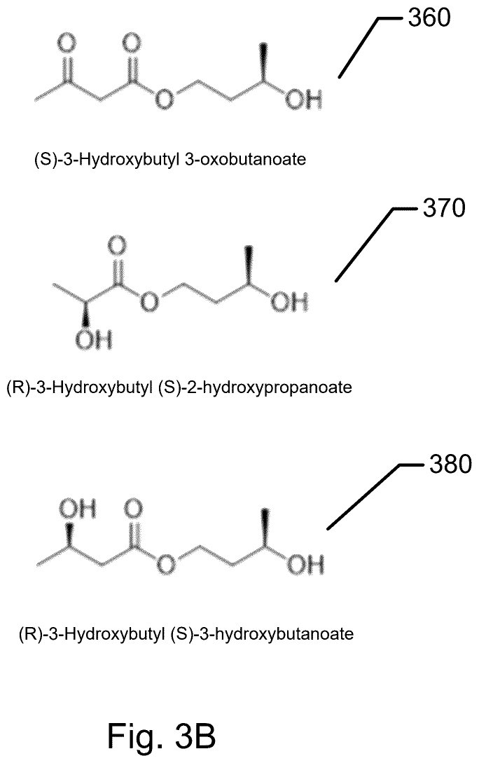

Referring to FIG. 3A, an exemplary set of six targeted primary esters is illustrated. These esters may include 3-Hydroxy-3-methylbutyl 3-oxobutanoate 300, (S)-3-Hydroxybutyl 3-oxobutanoate 310, (R)-3-Hydroxybutyl (S)-2-hydroxypropanoate 320, 3-Hydroxy-3-methylbutyl (S)-2-hydroxypropanoate 330, (R)-3-Hydroxybutyl (S)-3-hydroxybutanoate 340, and 3-Hydroxy-3-methylbutyl (S)-3-hydroxybutanoate 350.

Referring to FIG. 3B, an exemplary set of three targeted primary esters are illustrated. These may include (S)-3-Hydroxybutyl 3-oxobutanoate 360, (R)-3-Hydroxybutyl (S)-2-hydroxypropanoate 370, and (R)-3-Hydroxybutyl (S)-3-hydroxybutanoate 380.

The resulting purified mixtures may be ingested by a user.

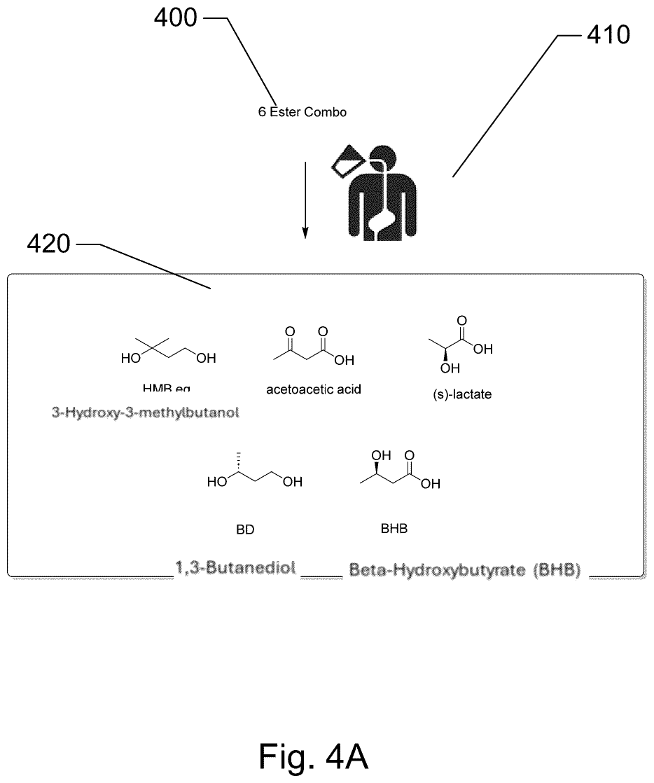

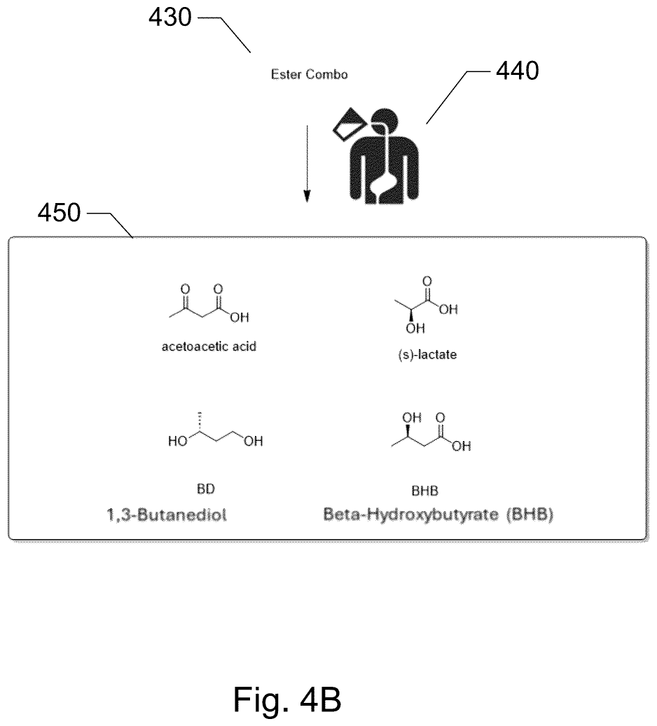

Referring now to FIG. 4A, a composition comprising a mixture with defined and definable relative amounts of the six targeted esters 400 may be ingested by a user 410. As the nutritional composition is metabolized in the digestive system of the user 410, various molecules 420 including the targeted molecules related to natural ketosis may be generated within the body of the user and make their way into the blood stream.

Referring now to FIG. 4B, in a similar illustration, a different composition with three targeted esters may be ingested by a user. As illustrated in FIG. 4B, the composition comprising a mixture with defined and definable relative amounts of the three targeted esters 430 may be ingested by a user 440. As the nutritional composition is metabolized in the digestive system of the user 440 various molecules 450, including the targeted molecules related to natural ketosis, may be generated within the body of the user and make their way into the blood stream.

In addition to the ketone esters specifically mentioned in the invention, other compounds that may be used to provide supplementation via ketone esters include:

Ethyl Acetoacetate: This compound can be used to synthesize ketone esters that release acetoacetate upon metabolism.

Ethyl β-Hydroxybutyrate: This ester can be metabolized to release β-hydroxybutyrate, a primary ketone body.

Acetoacetyl-CoA Derivatives: These can be used to form esters that release acetoacetate, contributing to ketosis.

Butanediol Monoesters: These compounds can be metabolized to produce β-hydroxybutyrate.

Glycerol Esters of Ketone Bodies: These esters can provide a sustained release of ketone bodies upon digestion.

Medium-Chain Triglycerides (MCTs): While not esters of ketone bodies, MCTs are rapidly metabolized to ketones in the liver and can be used in conjunction with ketone esters to enhance ketosis.

Propylene Glycol Esters: These can be used to deliver ketone bodies in a bioavailable form.

These compounds can be formulated to provide a controlled release of ketone bodies, supporting various health benefits associated with ketosis.

In some embodiments, the present disclosure provides a nutritional composition comprising a mixture of ester compounds that, upon ingestion and metabolism in a human gastrointestinal tract and/or liver, yields one or more ketosis-related molecules including acetoacetate (AcAc), beta-hydroxybutyrate (BHB), acetone, and/or metabolic precursors thereof. In some embodiments, the ester compounds are ketone body analog esters selected to provide, in vivo, a blood ketone profile that more closely resembles endogenous ketosis than administration of a single ketone body species.

In some embodiments, the composition comprises two or more esters selected from the group consisting of 3-hydroxy-3-methylbutyl 3-oxobutanoate, (S)-3-hydroxybutyl 3-oxobutanoate, (R)-3-hydroxybutyl (S)-2-hydroxypropanoate, 3-hydroxy-3-methylbutyl (S)-2-hydroxypropanoate, (R)-3-hydroxybutyl (S)-3-hydroxybutanoate, and 3-hydroxy-3-methylbutyl (S)-3-hydroxybutanoate. In some embodiments, the composition comprises 3-hydroxy-3-methylbutyl 3-oxobutanoate and (S)-3-hydroxybutyl 3-oxobutanoate. In some embodiments, the composition comprises (R)-3-hydroxybutyl (S)-2-hydroxypropanoate and 3-hydroxy-3-methylbutyl (S)-2-hydroxypropanoate. In some embodiments, the composition comprises (R)-3-hydroxybutyl (S)-3-hydroxybutanoate and 3-hydroxy-3-methylbutyl (S)-3-hydroxybutanoate. In some embodiments, the composition comprises three or more of the foregoing esters, including any combination of three, four, five, or all six of the foregoing esters, in any relative amounts effective to provide a desired post-ingestion ketone response.

In some embodiments, the mixture comprises the foregoing esters as “primary esters” formed by esterification of a primary hydroxyl of a diol and/or a primary hydroxyl of 3-hydroxy-3-methylbutanol. In some embodiments, the mixture further comprises one or more “secondary esters” formed at a secondary hydroxyl position of a polyol starting material, and such secondary esters may be present at a lower concentration relative to the primary esters due to steric and/or kinetic effects. In some embodiments, the disclosed compositions include any stereochemical form of a chiral ester component, including an enantiomerically enriched form, a racemate, or a diastereomeric mixture, and the disclosure of a particular stereochemical designation is intended to support, unless expressly limited, any stereoisomeric form and mixtures thereof.

In some embodiments, the composition is produced as a controlled product stream from a synthesis in which multiple reactants are combined in a single reaction vessel to form a mixture of esters via competing esterification and/or transesterification pathways. In some embodiments, the relative amounts of individual esters in the mixture are controlled by selecting (i) relative molar ratios of alcohol reactants to acid reactants, (ii) an identity and loading of a catalyst and/or enzyme system, (iii) reaction temperature, (iv) reaction time, (v) water activity and/or removal of water, and/or (vi) selection of solvent. In some embodiments, a target ester profile is achieved without isolating each individual ester to purity prior to formulation, thereby enabling a mixture intended to mimic aspects of natural ketosis.

In some embodiments, the methods of making comprise reacting 1,3-butanediol with one or more acids in the presence of an enzyme system to form one or more butanediol monoesters. In some embodiments, 1,3-butanediol is reacted with acetoacetic acid to form (R)-3-hydroxybutyl 3-oxobutanoate and/or (S)-3-hydroxybutyl 3-oxobutanoate, including mixtures thereof. In some embodiments, 1,3-butanediol is reacted with lactic acid to form (R)-3-hydroxybutyl (S)-2-hydroxypropanoate and/or stereoisomeric forms thereof. In some embodiments, 1,3-butanediol is reacted with beta-hydroxybutyric acid (including an enantiomerically enriched beta-hydroxybutyric acid) to form (R)-3-hydroxybutyl (S)-3-hydroxybutanoate and/or stereoisomeric forms thereof. In some embodiments, two or more of acetoacetic acid, lactic acid, and beta-hydroxybutyric acid are concurrently present with 1,3-butanediol in a single reaction to form a mixture comprising two or more corresponding butanediol monoesters.

In some embodiments, the methods of making comprise reacting 3-hydroxy-3-methylbutanol with one or more acids in the presence of an enzyme system to form one or more 3-hydroxy-3-methylbutanol esters. In some embodiments, 3-hydroxy-3-methylbutanol is reacted with acetoacetic acid to form 3-hydroxy-3-methylbutyl 3-oxobutanoate. In some embodiments, 3-hydroxy-3-methylbutanol is reacted with lactic acid to form 3-hydroxy-3-methylbutyl (S)-2-hydroxypropanoate and/or stereoisomeric forms thereof. In some embodiments, 3-hydroxy-3-methylbutanol is reacted with beta-hydroxybutyric acid to form 3-hydroxy-3-methylbutyl (S)-3-hydroxybutanoate and/or stereoisomeric forms thereof. In some embodiments, two or more of acetoacetic acid, lactic acid, and beta-hydroxybutyric acid are concurrently present with 3-hydroxy-3-methylbutanol in a single reaction to form a mixture comprising two or more corresponding esters.

In some embodiments, the methods of making comprise combining 1,3-butanediol, 3-hydroxy-3-methylbutanol, and two or more acids selected from acetoacetic acid, lactic acid, and beta-hydroxybutyric acid in a single reaction in the presence of an enzyme system, thereby producing a mixture comprising two or more of the following: 3-hydroxy-3-methylbutyl 3-oxobutanoate, (S)-3-hydroxybutyl 3-oxobutanoate, (R)-3-hydroxybutyl (S)-2-hydroxypropanoate, 3-hydroxy-3-methylbutyl (S)-2-hydroxypropanoate, (R)-3-hydroxybutyl (S)-3-hydroxybutanoate, and 3-hydroxy-3-methylbutyl (S)-3-hydroxybutanoate. In some embodiments, the reaction produces a mixture comprising at least three of the foregoing esters, and in some embodiments at least four of the foregoing esters, and in some embodiments all six of the foregoing esters, optionally together with secondary esters and/or residual unreacted starting materials within acceptable ingestion limits.

In some embodiments, the enzyme system comprises a lipase catalyst. In some embodiments, the lipase is provided as a free enzyme or as an immobilized enzyme on a solid support. In some embodiments, the reaction is conducted in a solvent system comprising one or more of water, ethyl acetate, toluene, and xylene, and/or in a solvent-free melt. In some embodiments, the reaction is conducted at a temperature above room temperature. In some embodiments, the reaction is conducted at a temperature in a range from about 20° C. to about 150° C. In some embodiments, the reaction is conducted in the presence of an acid catalyst or a base catalyst, including sulfuric acid and/or triethylamine, and the catalyst may be used in addition to or in place of the enzyme system. In some embodiments, the product mixture is used without full separation of each ester component. In some embodiments, purification and/or refinement comprises extraction, distillation, column chromatography, and/or other separation operations suitable to remove catalyst residues, solvent residues, and undesired byproducts.

In some embodiments, the present disclosure further provides methods of providing supplementation to a user by administering an effective amount of any of the compositions described herein, including compositions comprising one ester, two esters, three esters, or more, and including compositions comprising a defined and definable mixture profile of the disclosed esters. In some embodiments, administration increases a ketone body level in a bloodstream relative to a pre-administration baseline. In some embodiments, the ketone body level comprises a BHB level, an AcAc level, a total ketone level, and/or a breath acetone level. In some embodiments, the composition is administered as a dietary supplement, medical food, food product, beverage product, powder, capsule, tablet, gel, or liquid, and may further comprise one or more excipients, flavoring agents, sweeteners, stabilizers, emulsifiers, and/or carriers suitable for oral administration.

In some embodiments, administering a composition comprising 3-hydroxy-3-methylbutyl 3-oxobutanoate increases ketone body levels in the bloodstream. In some embodiments, administering a composition comprising (S)-3-hydroxybutyl 3-oxobutanoate supports energy metabolism by providing an exogenous ketone precursor load. In some embodiments, administering a composition comprising (R)-3-hydroxybutyl (S)-2-hydroxypropanoate supports muscle recovery and/or exercise performance, including by providing an alternative oxidative fuel substrate. In some embodiments, administering a composition comprising 3-hydroxy-3-methylbutyl (S)-2-hydroxypropanoate supports cognitive function and/or mental energy. In some embodiments, administering a composition comprising (R)-3-hydroxybutyl (S)-3-hydroxybutanoate supports weight management and/or metabolic health. In some embodiments, administering a composition comprising 3-hydroxy-3-methylbutyl (S)-3-hydroxybutanoate supports cardiovascular health and/or endurance performance. In some embodiments, administering a composition comprising mixtures of two or more of the foregoing esters promotes overall wellness by providing multiple ketosis-related molecules and/or precursors over a time course following ingestion.

In some embodiments, the disclosed compositions optionally further comprise one or more additional ketone-related compounds, including ethyl acetoacetate, ethyl beta-hydroxybutyrate, glycerol esters of ketone bodies, butanediol monoesters, propylene glycol esters, medium-chain triglycerides, and combinations thereof, thereby enabling tuning of onset, magnitude, and duration of a ketone response and/or gastrointestinal tolerance. In some embodiments, the disclosed compositions are formulated such that the ester mixture profile and optional additional ketone-related compounds provide a controlled release and/or sustained generation of ketone bodies upon digestion.

In some embodiments, the present disclosure provides a composition comprising a mixture of esters, wherein the esters include 3-Hydroxy-3-methylbutyl 3-oxobutanoate and(S)-3-Hydroxybutyl 3-oxobutanoate. In such embodiments, the mixture may be formulated and/or produced such that both ester species are present in the same ingestible composition as co-components of the mixture.

In some embodiments, the present disclosure provides a composition comprising a mixture of esters, wherein the esters include (R)-3-Hydroxybutyl (S)-2-hydroxypropanoate and 3-Hydroxy-3-methylbutyl (S)-2-hydroxypropanoate. In such embodiments, the mixture may be formulated and/or produced such that both ester species are present in the same ingestible composition as co-components of the mixture.

In some embodiments, the present disclosure provides a composition comprising a mixture of esters, wherein the esters include (R)-3-Hydroxybutyl (S)-3-hydroxybutanoate and 3-Hydroxy-3-methylbutyl (S)-3-hydroxybutanoate. In such embodiments, the mixture may be formulated and/or produced such that both ester species are present in the same ingestible composition as co-components of the mixture.

In some embodiments, the present disclosure provides a composition comprising a mixture of esters, wherein the esters include 3-Hydroxy-3-methylbutyl 3-oxobutanoate, (S)-3-Hydroxybutyl 3-oxobutanoate, and (R)-3-Hydroxybutyl (S)-2-hydroxypropanoate. In such embodiments, the mixture includes the recited three ester species together in the same composition, optionally with additional ester species and/or secondary ester species.

In some embodiments, the present disclosure provides a composition comprising a mixture of esters, wherein the esters include 3-Hydroxy-3-methylbutyl (S)-2-hydroxypropanoate, (R)-3-Hydroxybutyl (S)-3-hydroxybutanoate, and 3-Hydroxy-3-methylbutyl (S)-3-hydroxybutanoate. In such embodiments, the mixture includes the recited three ester species together in the same composition, optionally with additional ester species and/or secondary ester species.

In some embodiments, the present disclosure provides a composition comprising a mixture of esters, wherein the esters include (S)-3-Hydroxybutyl 3-oxobutanoate, (R)-3-Hydroxybutyl (S)-2-hydroxypropanoate, and (R)-3-Hydroxybutyl (S)-3-hydroxybutanoate. In such embodiments, the mixture includes the recited three ester species together in the same composition, optionally with additional ester species and/or secondary ester species.

In some embodiments, the present disclosure provides a composition comprising a mixture of esters, wherein the esters include 3-Hydroxy-3-methylbutyl 3-oxobutanoate, (S)-3-Hydroxybutyl 3-oxobutanoate, and 3-Hydroxy-3-methylbutyl (S)-2-hydroxypropanoate. In such embodiments, the mixture includes the recited three ester species together in the same composition, optionally with additional ester species and/or secondary ester species.

In some embodiments, the present disclosure provides a composition comprising a mixture of esters, wherein the esters include (R)-3-Hydroxybutyl (S)-2-hydroxypropanoate, 3-Hydroxy-3-methylbutyl (S)-3-hydroxybutanoate, and (S)-3-Hydroxybutyl 3-oxobutanoate. In such embodiments, the mixture includes the recited three ester species together in the same composition, optionally with additional ester species and/or secondary ester species.

In some embodiments, the present disclosure provides a composition comprising a mixture of esters, wherein the esters include 3-Hydroxy-3-methylbutyl 3-oxobutanoate, (R)-3-Hydroxybutyl (S)-3-hydroxybutanoate, and (S)-3-Hydroxybutyl 3-oxobutanoate. In such embodiments, the mixture includes the recited three ester species together in the same composition, optionally with additional ester species and/or secondary ester species.

In some embodiments, the present disclosure provides a composition comprising a mixture of esters, wherein the esters include (R)-3-Hydroxybutyl (S)-2-hydroxypropanoate, 3-Hydroxy-3-methylbutyl 3-oxobutanoate, and 3-Hydroxy-3-methylbutyl (S)-3-hydroxybutanoate. In such embodiments, the mixture includes the recited three ester species together in the same composition, optionally with additional ester species and/or secondary ester species.

In some embodiments, the present disclosure provides a composition comprising a mixture of esters, wherein the esters include 3-Hydroxy-3-methylbutyl 3-oxobutanoate, (S)-3-Hydroxybutyl 3-oxobutanoate, (R)-3-Hydroxybutyl (S)-3-hydroxybutanoate, and 3-Hydroxy-3-methylbutyl (S)-2-hydroxypropanoate. In such embodiments, the mixture includes the recited four ester species together in the same composition, optionally with additional ester species and/or secondary ester species.

In some embodiments, the present disclosure provides a composition comprising a mixture of esters, wherein the esters include (R)-3-Hydroxybutyl (S)-2-hydroxypropanoate, 3-Hydroxy-3-methylbutyl 3-oxobutanoate, (S)-3-Hydroxybutyl 3-oxobutanoate, and 3-Hydroxy-3-methylbutyl (S)-3-hydroxybutanoate. In such embodiments, the mixture includes the recited four ester species together in the same composition, optionally with additional ester species and/or secondary ester species.

In some embodiments, the present disclosure provides a composition comprising a mixture of esters, wherein the esters include 3-Hydroxy-3-methylbutyl 3-oxobutanoate, (R)-3-Hydroxybutyl (S)-3-hydroxybutanoate, (S)-3-Hydroxybutyl 3-oxobutanoate, and (R)-3-Hydroxybutyl (S)-2-hydroxypropanoate. In such embodiments, the mixture includes the recited four ester species together in the same composition, optionally with additional ester species and/or secondary ester species.

In some embodiments, the present disclosure provides a composition comprising a mixture of esters, wherein the esters include 3-Hydroxy-3-methylbutyl 3-oxobutanoate, (S)-3-Hydroxybutyl 3-oxobutanoate, (R)-3-Hydroxybutyl (S)-2-hydroxypropanoate, and 3-Hydroxy-3-methylbutyl (S)-3-hydroxybutanoate. In such embodiments, the mixture includes the recited four ester species together in the same composition, optionally with additional ester species and/or secondary ester species.

In some embodiments, the present disclosure provides a composition comprising a mixture of esters, wherein the esters include (R)-3-Hydroxybutyl (S)-3-hydroxybutanoate, 3-Hydroxy-3-methylbutyl 3-oxobutanoate, (S)-3-Hydroxybutyl 3-oxobutanoate, and (R)-3-Hydroxybutyl (S)-2-hydroxypropanoate. In such embodiments, the mixture includes the recited four ester species together in the same composition, optionally with additional ester species and/or secondary ester species.

In some embodiments, the present disclosure provides a composition comprising a mixture of esters, wherein the esters include 3-Hydroxy-3-methylbutyl 3-oxobutanoate, (S)-3-Hydroxybutyl 3-oxobutanoate, (R)-3-Hydroxybutyl (S)-3-hydroxybutanoate, and 3-Hydroxy-3-methylbutyl (S)-2-hydroxypropanoate. In such embodiments, the mixture includes the recited four ester species together in the same composition, optionally with additional ester species and/or secondary ester species.

In some embodiments, the present disclosure provides a composition comprising a mixture of esters, wherein the esters include (R)-3-Hydroxybutyl (S)-2-hydroxypropanoate, 3-Hydroxy-3-methylbutyl 3-oxobutanoate, (S)-3-Hydroxybutyl 3-oxobutanoate, and (R)-3-Hydroxybutyl (S)-3-hydroxybutanoate. In such embodiments, the mixture includes the recited four ester species together in the same composition, optionally with additional ester species and/or secondary ester species.

In some embodiments, the present disclosure provides a composition comprising a mixture of esters, wherein the esters include 3-Hydroxy-3-methylbutyl 3-oxobutanoate, (R)-3-Hydroxybutyl (S)-3-hydroxybutanoate, (S)-3-Hydroxybutyl 3-oxobutanoate, and (R)-3-Hydroxybutyl (S)-2-hydroxypropanoate. In such embodiments, the mixture includes the recited four ester species together in the same composition, optionally with additional ester species and/or secondary ester species.

In some embodiments, the present disclosure provides a composition comprising a mixture of esters, wherein the esters include 3-Hydroxy-3-methylbutyl 3-oxobutanoate, (S)-3-Hydroxybutyl 3-oxobutanoate, (R)-3-Hydroxybutyl (S)-2-hydroxypropanoate, and 3-Hydroxy-3-methylbutyl (S)-3-hydroxybutanoate. In such embodiments, the mixture includes the recited four ester species together in the same composition, optionally with additional ester species and/or secondary ester species.

In some embodiments, the present disclosure provides a composition comprising a mixture of esters, wherein the esters include (R)-3-Hydroxybutyl (S)-3-hydroxybutanoate, 3-Hydroxy-3-methylbutyl 3-oxobutanoate, (S)-3-Hydroxybutyl 3-oxobutanoate, and (R)-3-Hydroxybutyl (S)-2-hydroxypropanoate. In such embodiments, the mixture includes the recited four ester species together in the same composition, optionally with additional ester species and/or secondary ester species.

In some embodiments, each of the foregoing compositions comprising a mixture of esters is produced by forming at least a portion of the mixture in a chemical reaction, and optionally completing the mixture by blending. In some embodiments, 3-Hydroxy-3-methylbutyl 3-oxobutanoate is formed by reacting 3-Hydroxy-3-methylbutanol with acetoacetic acid in a presence of an enzyme system. In some embodiments, (S)-3-Hydroxybutyl 3-oxobutanoate is formed by reacting 1,3-Butanediol with acetoacetic acid in a presence of an enzyme system. In some embodiments, (R)-3-Hydroxybutyl (S)-2-hydroxypropanoate is formed by reacting 1,3-Butanediol with lactic acid in a presence of an enzyme system. In some embodiments, 3-Hydroxy-3-methylbutyl (S)-2-hydroxypropanoate is formed by reacting 3-Hydroxy-3-methylbutanol with lactic acid in a presence of an enzyme system. In some embodiments, (R)-3-Hydroxybutyl (S)-3-hydroxybutanoate is formed by reacting 1,3-Butanediol with Beta-Hydroxybutyric acid in a presence of an enzyme system. In some embodiments, 3-Hydroxy-3-methylbutyl (S)-3-hydroxybutanoate is formed by reacting 3-Hydroxy-3-methylbutanol with Beta-Hydroxybutyric acid in a presence of an enzyme system.

In some embodiments, the mixture of esters is produced in a single reaction vessel by combining two or more alcohol reactants and two or more acid reactants in a presence of an enzyme system, thereby forming a plurality of ester products that collectively constitute the mixture of esters, and optionally followed by purification to remove catalyst, solvent, and/or undesired byproducts. In some embodiments, combining 1,3-Butanediol, 3-Hydroxy-3-methylbutanol, and acetoacetic acid in a presence of an enzyme system forms a product mixture that includes 3-Hydroxy-3-methylbutyl 3-oxobutanoate and (S)-3-Hydroxybutyl 3-oxobutanoate. In some embodiments, combining 1,3-Butanediol, 3-Hydroxy-3-methylbutanol, and lactic acid in a presence of an enzyme system forms a product mixture that includes (R)-3-Hydroxybutyl (S)-2-hydroxypropanoate and 3-Hydroxy-3-methylbutyl (S)-2-hydroxypropanoate. In some embodiments, combining 1,3-Butanediol, 3-Hydroxy-3-methylbutanol, and Beta-Hydroxybutyric acid in a presence of an enzyme system forms a product mixture that includes (R)-3-Hydroxybutyl (S)-3-hydroxybutanoate and 3-Hydroxy-3-methylbutyl (S)-3-hydroxybutanoate.

In some embodiments, combining 1,3-Butanediol, acetoacetic acid, and lactic acid in a presence of an enzyme system forms a product mixture that includes(S)-3-Hydroxybutyl 3-oxobutanoate and (R)-3-Hydroxybutyl (S)-2-hydroxypropanoate, and the product mixture may optionally further include additional ester species if additional reactants are present or if transesterification occurs. In some embodiments, combining 1,3-Butanediol, acetoacetic acid, and Beta-Hydroxybutyric acid in a presence of an enzyme system forms a product mixture that includes (S)-3-Hydroxybutyl 3-oxobutanoate and (R)-3-Hydroxybutyl (S)-3-hydroxybutanoate. In some embodiments, combining 1,3-Butanediol, lactic acid, and Beta-Hydroxybutyric acid in a presence of an enzyme system forms a product mixture that includes (R)-3-Hydroxybutyl (S)-2-hydroxypropanoate and (R)-3-Hydroxybutyl (S)-3-hydroxybutanoate.

In some embodiments, combining 3-Hydroxy-3-methylbutanol, acetoacetic acid, and lactic acid in a presence of an enzyme system forms a product mixture that includes 3-Hydroxy-3-methylbutyl 3-oxobutanoate and 3-Hydroxy-3-methylbutyl (S)-2-hydroxypropanoate. In some embodiments, combining 3-Hydroxy-3-methylbutanol, acetoacetic acid, and Beta-Hydroxybutyric acid in a presence of an enzyme system forms a product mixture that includes 3-Hydroxy-3-methylbutyl 3-oxobutanoate and 3-Hydroxy-3-methylbutyl (S)-3-hydroxybutanoate. In some embodiments, combining 3-Hydroxy-3-methylbutanol, lactic acid, and Beta-Hydroxybutyric acid in a presence of an enzyme system forms a product mixture that includes 3-Hydroxy-3-methylbutyl (S)-2-hydroxypropanoate and 3-Hydroxy-3-methylbutyl (S)-3-hydroxybutanoate.

In some embodiments, combining acetoacetic acid, lactic acid, and Beta-Hydroxybutyric acid in a presence of an enzyme system is performed together with at least one alcohol reactant, and in some embodiments is performed together with 1,3-Butanediol and/or 3-Hydroxy-3-methylbutanol, to form a product mixture that includes one or more esters derived from one or more of the acids, including one or more of 3-Hydroxy-3-methylbutyl 3-oxobutanoate, (S)-3-Hydroxybutyl 3-oxobutanoate, (R)-3-Hydroxybutyl (S)-2-hydroxypropanoate, 3-Hydroxy-3-methylbutyl (S)-2-hydroxypropanoate, (R)-3-Hydroxybutyl (S)-3-hydroxybutanoate, and 3-Hydroxy-3-methylbutyl (S)-3-hydroxybutanoate.

In some embodiments, combining 1,3-Butanediol, acetoacetic acid, lactic acid, and Beta-Hydroxybutyric acid in a presence of an enzyme system forms a product mixture that includes(S)-3-Hydroxybutyl 3-oxobutanoate, (R)-3-Hydroxybutyl (S)-2-hydroxypropanoate, and (R)-3-Hydroxybutyl (S)-3-hydroxybutanoate. In some embodiments, combining 3-Hydroxy-3-methylbutanol, acetoacetic acid, lactic acid, and Beta-Hydroxybutyric acid in a presence of an enzyme system forms a product mixture that includes 3-Hydroxy-3-methylbutyl 3-oxobutanoate, 3-Hydroxy-3-methylbutyl (S)-2-hydroxypropanoate, and 3-Hydroxy-3-methylbutyl (S)-3-hydroxybutanoate. In some embodiments, combining 1,3-Butanediol, 3-Hydroxy-3-methylbutanol, acetoacetic acid, and lactic acid in a presence of an enzyme system forms a product mixture that includes 3-Hydroxy-3-methylbutyl 3-oxobutanoate, (S)-3-Hydroxybutyl 3-oxobutanoate, (R)-3-Hydroxybutyl (S)-2-hydroxypropanoate, and 3-Hydroxy-3-methylbutyl (S)-2-hydroxypropanoate. In some embodiments, combining 1,3-Butanediol, 3-Hydroxy-3-methylbutanol, acetoacetic acid, and Beta-Hydroxybutyric acid in a presence of an enzyme system forms a product mixture that includes 3-Hydroxy-3-methylbutyl 3-oxobutanoate, (S)-3-Hydroxybutyl 3-oxobutanoate, (R)-3-Hydroxybutyl (S)-3-hydroxybutanoate, and 3-Hydroxy-3-methylbutyl (S)-3-hydroxybutanoate.

In some embodiments, the enzyme system comprises a lipase, optionally immobilized, and the reaction conditions are selected such that the resulting product mixture comprises the recited ester components in relative amounts suitable for oral administration as a dietary supplement. In some embodiments, after forming the product mixture, one or more ester components are adjusted by blending additional quantities of one or more of 3-Hydroxy-3-methylbutyl 3-oxobutanoate, (S)-3-Hydroxybutyl 3-oxobutanoate, (R)-3-Hydroxybutyl (S)-2-hydroxypropanoate, 3-Hydroxy-3-methylbutyl (S)-2-hydroxypropanoate, (R)-3-Hydroxybutyl (S)-3-hydroxybutanoate, and 3-Hydroxy-3-methylbutyl (S)-3-hydroxybutanoate into the mixture, thereby yielding a final composition comprising a mixture of esters that includes any of the recited combinations.

In certain embodiments, the present disclosure provides enzymatic methods for synthesizing ester-containing compositions through biocatalytic esterification and/or transesterification reactions between one or more alcohol substrates and one or more carboxylic acid substrates. The alcohol substrates may include 1,3-butanediol, which may be present as a racemic mixture or as an enriched (R)- or(S)-enantiomer, and/or 3-hydroxy-3-methylbutanol. The acid substrates may include acetoacetic acid (3-oxobutanoic acid), lactic acid (2-hydroxypropanoic acid), and beta-hydroxybutyric acid (3-hydroxybutanoic acid), each of which may independently be provided as a racemate or as a stereochemically enriched form. Ester formation is catalyzed in the presence of an enzyme system capable of promoting condensation between hydroxyl and carboxyl functional groups under mild conditions.

Suitable enzyme systems include lipases, esterases, cutinases, acyltransferases, and combinations thereof. In certain embodiments, the enzyme system comprises an immobilized lipase, such as a Candida antarctica lipase B (CALB), Thermomyces lanuginosus lipase, Rhizomucor miehei lipase, or a genetically engineered variant thereof exhibiting enhanced selectivity for secondary alcohol substrates. Immobilization may be achieved on polymeric resins, silica supports, magnetic particles, or other solid carriers to facilitate enzyme recovery and reuse. In some embodiments, the enzyme system is provided in free form, encapsulated form, cross-linked enzyme aggregates (CLEAs), or whole-cell biocatalyst form.

Esterification reactions may be conducted in solvent-free systems or in organic solvents such as hexane, heptane, tert-butanol, acetonitrile, or ethyl acetate. In other embodiments, the reaction is performed in a biphasic aqueous/organic system. Reaction temperatures may range from about 20° C. to about 80° C., and in particular embodiments from about 30° C. to about 60° C. The reaction pH, when conducted in aqueous media, may range from about pH 4 to about pH 8. Water activity may be controlled to favor ester formation, including through molecular sieves, reduced pressure, nitrogen sparging, or continuous removal of water formed during the reaction. Substrate molar ratios may be adjusted to drive conversion, for example by using an excess of alcohol or acid. Reaction times may range from several hours to several days depending on substrate loading and enzyme activity.

In embodiments corresponding to claims 21-26, a single alcohol substrate (1,3-butanediol or 3-hydroxy-3-methylbutanol) is reacted with a single acid substrate (acetoacetic acid, lactic acid, or beta-hydroxybutyric acid) to yield the corresponding monoester and, where applicable, diester products. For example, reacting 1,3-butanediol with acetoacetic acid in the presence of a lipase enzyme system produces 3-hydroxybutyl 3-oxobutanoate, which may further undergo selective esterification at the second hydroxyl group depending on stoichiometry and reaction conditions. Similarly, reaction with lactic acid yields 3-hydroxybutyl 2-hydroxypropanoate, and reaction with beta-hydroxybutyric acid yields 3-hydroxybutyl 3-hydroxybutanoate. When 3-hydroxy-3-methylbutanol is used as the alcohol substrate, corresponding 3-hydroxy-3-methylbutyl esters are formed under analogous conditions.

In embodiments corresponding to claims 27-40, two or more substrates are combined in a single reaction system to produce mixed ester compositions. For example, combinations of 1,3-butanediol with acetoacetic acid and lactic acid may yield mixtures comprising 3-hydroxybutyl 3-oxobutanoate and 3-hydroxybutyl 2-hydroxypropanoate. Inclusion of beta-hydroxybutyric acid introduces formation of 3-hydroxybutyl 3-hydroxybutanoate. When both alcohol substrates (1,3-butanediol and 3-hydroxy-3-methylbutanol) are present with one or more acid substrates, the enzyme system catalyzes parallel esterification reactions, generating mixtures of corresponding butyl and 3-hydroxy-3-methylbutyl esters. Relative product distribution may be controlled by adjusting substrate molar ratios, enzyme selectivity, stereochemical composition, temperature, and reaction time. In certain embodiments, sequential addition of substrates is employed to preferentially form a first ester before introducing additional acid or alcohol components to generate defined mixtures.

Stereochemical control may be achieved by selecting enantiomerically enriched substrates and/or stereoselective enzymes. For example, use of (S)-lactic acid with (R)-1,3-butanediol in the presence of an enantioselective lipase may preferentially yield (R)-3-hydroxybutyl (S)-2-hydroxypropanoate. Similarly, use of (S)-beta-hydroxybutyric acid may yield stereochemically defined 3-hydroxybutyl (S)-3-hydroxybutanoate. Enzymatic catalysis under mild conditions preserves stereochemical integrity and minimizes racemization of chiral centers.

Following synthesis, the ester-containing composition may be purified by techniques including vacuum distillation, fractional distillation, liquid-liquid extraction, crystallization, preparative chromatography, or combinations thereof. In some embodiments, the reaction mixture is filtered to remove immobilized enzyme, washed to remove unreacted acid, dried, and concentrated to yield a purified ester product. Analytical confirmation may be performed using gas chromatography (GC), high-performance liquid chromatography (HPLC), nuclear magnetic resonance (NMR) spectroscopy, mass spectrometry (MS), and chiral chromatography where applicable.

In certain embodiments corresponding to claims 41-50, the synthesized esters are incorporated into supplementation compositions suitable for administration to a user. The compositions may be formulated as liquids, emulsions, capsules, softgels, powders, suspensions, or controlled-release dosage forms. Excipients may include carriers such as medium-chain triglycerides (MCT oil), glycerol, propylene glycol, ethanol, or aqueous buffers. Flavoring agents, stabilizers, antioxidants, and preservatives may be included as appropriate.

Upon administration, esters such as 3-hydroxy-3-methylbutyl 3-oxobutanoate and(S)-3-hydroxybutyl 3-oxobutanoate may undergo in vivo hydrolysis by endogenous esterases to release corresponding ketone body precursors, including acetoacetate and beta-hydroxybutyrate, thereby increasing circulating ketone levels in the bloodstream. Esters comprising lactic acid moieties may release lactate, supporting energy metabolism through gluconeogenic or oxidative pathways. Compositions comprising beta-hydroxybutyrate esters may provide substrates for mitochondrial oxidation and ATP production, thereby supporting energy metabolism, endurance performance, muscle recovery, weight management, cardiovascular function, immune support, cognitive function, and overall metabolic health.

Dosage amounts may range, in certain embodiments, from about 1 gram to about 50 grams per day of total ester content, administered in one or more divided doses. The specific dosage may depend on body weight, metabolic status, and intended physiological effect. In some embodiments, mixtures of esters as recited in claims 47-50 provide synergistic metabolic effects by delivering multiple ketone and hydroxy acid precursors simultaneously.

The enzymatic synthesis methods described herein may provide advantages over conventional chemical esterification, such as, for example: milder reaction conditions, reduced byproduct formation, stereochemical selectivity, lower energy consumption, and suitability for food-grade or nutraceutical production. The disclosed methods enable preparation of single esters or tailored mixtures suitable for incorporation into supplementation compositions designed to modulate metabolic pathways in a user.

It will be evident to one skilled in the art that the present invention is not limited to the foregoing illustrative examples, and that it can be embodied in other specific forms without departing from the essential attributes thereof. It is therefore desired that the examples be considered in all respects as illustrative and not restrictive, reference being made to the appended claims, rather than to the foregoing examples, and all changes which come within the meaning and range of equivalency of the claims are therefore intended to be embraced therein.

While specific combinations of compounds are disclosed in specific embodiments, it should be understood that there exist numerous combinations of the different compound which may be utilized in the compound of the formulas for the present invention.

The foregoing disclosure and description of the invention are illustrative and explanatory thereof, and various changes in the compounds of illustrated formulas may be changed without departing from the spirit of the invention.

It is understood that the invention is not limited to the specific embodiments disclosed and that modifications and other embodiments are intended to be included within the scope of the appended claims. Although specific terms are employed herein, they are used in a generic and descriptive sense only and not for purposes of limitation.

What is claimed is:

1. A composition comprising a mixture of esters, wherein the esters include 3-Hydroxy-3-methylbutyl 3-oxobutanoate and (S)-3-Hydroxybutyl 3-oxobutanoate.

2. The composition of claim 1, wherein the mixture of esters further comprises (R)-3-Hydroxybutyl (S)-2-hydroxypropanoate.

3. The composition of claim 1, wherein the mixture of esters further comprises 3-Hydroxy-3-methylbutyl (S)-2-hydroxypropanoate.

4. The composition of claim 1, wherein the mixture of esters further comprises (R)-3-Hydroxybutyl (S)-3-hydroxybutanoate.

5. The composition of claim 1, wherein the mixture of esters further comprises 3-Hydroxy-3-methylbutyl (S)-3-hydroxybutanoate.

6. The composition of claim 1, wherein a weight ratio of the 3-Hydroxy-3-methylbutyl 3-oxobutanoate to the (S)-3-Hydroxybutyl 3-oxobutanoate is from 0.1:1 to 10:1.

7. The composition of claim 1, wherein the composition is formulated for oral administration as a dietary supplement in a unit dose form selected from a capsule, a softgel, a tablet, a powder, a liquid, or a beverage.

8. A composition comprising a mixture of esters, wherein the mixture of esters comprises at least two esters selected from the group consisting of 3-Hydroxy-3-methylbutyl 3-oxobutanoate, (S)-3-Hydroxybutyl 3-oxobutanoate, (R)-3-Hydroxybutyl (S)-2-hydroxypropanoate, 3-Hydroxy-3-methylbutyl (S)-2-hydroxypropanoate, (R)-3-Hydroxybutyl (S)-3-hydroxybutanoate, and 3-Hydroxy-3-methylbutyl (S)-3-hydroxybutanoate.

9. The composition of claim 8, wherein the mixture of esters comprises at least three esters selected from the group.

10. The composition of claim 8, wherein the mixture of esters comprises 3-Hydroxy-3-methylbutyl 3-oxobutanoate, (S)-3-Hydroxybutyl 3-oxobutanoate, and (R)-3-Hydroxybutyl (S)-2-hydroxypropanoate.

11. The composition of claim 8, wherein the mixture of esters comprises (S)-3-Hydroxybutyl 3-oxobutanoate, (R)-3-Hydroxybutyl (S)-2-hydroxypropanoate, and (R)-3-Hydroxybutyl (S)-3-hydroxybutanoate.

12. The composition of claim 8, wherein the mixture of esters comprises 3-Hydroxy-3-methylbutyl 3-oxobutanoate, (S)-3-Hydroxybutyl 3-oxobutanoate, (R)-3-Hydroxybutyl (S)-2-hydroxypropanoate, and 3-Hydroxy-3-methylbutyl (S)-3-hydroxybutanoate.

13. The composition of claim 8, further comprising medium-chain triglycerides.

14. The composition of claim 8, wherein the composition comprises an excipient system comprising at least one of a flavoring agent, a sweetener, an emulsifier, or a stabilizer.

15. A composition comprising a mixture of esters, wherein the esters include (R)-3-Hydroxybutyl (S)-2-hydroxypropanoate and 3-Hydroxy-3-methylbutyl (S)-2-hydroxypropanoate.

16. The composition of claim 15, wherein the mixture of esters further comprises 3-Hydroxy-3-methylbutyl 3-oxobutanoate.

17. The composition of claim 15, wherein the mixture of esters further comprises (S)-3-Hydroxybutyl 3-oxobutanoate.

18. The composition of claim 15, wherein the mixture of esters further comprises (R)-3-Hydroxybutyl (S)-3-hydroxybutanoate.

19. The composition of claim 15, wherein the mixture of esters further comprises 3-Hydroxy-3-methylbutyl (S)-3-hydroxybutanoate.

20. The composition of claim 15, wherein a combined amount of (R)-3-Hydroxybutyl (S)-2-hydroxypropanoate and 3-Hydroxy-3-methylbutyl (S)-2-hydroxypropanoate is at least 50 wt. % of total esters in the mixture of esters.
